(19) United States
(12) Patent Application Publication
(10) Pub. No.: US 2013/0210794 A1
(43) Pub. Date: Aug. 15, 2013

(54) NANOSTRUCTURED EZETIMIBE COMPOSITIONS, PROCESS FOR THE PREPARATION THEREOF AND PHARMACEUTICAL COMPOSITIONS CONTAINING THEM

(71) Applicants: Genovéva Filipcsei, Budapest (HU); Zsolt Ötvös, Csongrad (HU); Gábor Heltovics, Budapest (HU); Ferenc Darvas, Budapest (HU)

(72) Inventors: Genovéva Filipcsei, Budapest (HU); Zsolt Ötvös, Csongrad (HU); Gábor Heltovics, Budapest (HU); Ferenc Darvas, Budapest (HU)

(21) Appl. No.: 13/703,843

(22) PCT Filed: Jun. 17, 2011
(86) PCT No.: PCT/HU11/00056

§ 371 Date: Jan. 23, 2013

(30) Foreign Application Priority Data
Jun. 18, 2010 (HU) P1000327

(51) Int. Cl. A61K 31/397

(52) U.S. Cl. CPC A61K 31/397
USPC 514/210.02

(57) ABSTRACT

The present invention is directed to nanostructured Ezetimibe compositions, process for the preparation thereof and pharmaceutical compositions containing them. The nanoparticles of Ezetimibe according to the invention have an average particle size of less than about 400 nm. The stable nanostructured particles of the invention are presented by increased solubility, dissolution rate, permeability and bioequivalent or enhanced biological performance compared to the marketed drug. Ezetimibe is an anti-hyperlipidemic medication that is used to lower cholesterol levels. It acts by decreasing cholesterol absorption in the intestine.

### FIELD OF THE INVENTION

The present invention is directed to nanostructured Ezetimibe compositions, process for the preparation thereof and pharmaceutical compositions containing them.

The nanoparticles of Ezetimibe according to the invention have an average particle size of less than about 400 nm. The stable nanostructured particles of the invention are presented by increased solubility, dissolution rate, permeability and bioequivalent or enhanced biological performance compared to the reference active compound. Ezetimibe is an anti-hyperlipidemic medication that is used to lower cholesterol levels. It acts by decreasing cholesterol absorption in the intestine.

### BACKGROUND OF THE INVENTION

### A. Background Regarding to Nanoparticle Formation/Production

Nowadays, the active ingredient developers run out of new chemical entities with high solubility; most compounds that are approved or enter development processes are poorly soluble and/or have low permeability. The traditional approaches to increase the solubility and dissolution rate of these compounds are very limited. Chemical modification, like salt- or prodrug formation and inclusion of ionizable groups could result in higher performance of the active compounds. However, these structural modifications can lead to inactivity or instability of the active compounds in many cases. Conventional solid or liquid formulations (e.g.; micronization, milling, solid dispersion, liposomes) could also be useful tools for the researchers to increase the solubility of the compounds, but the efficiency of the formulation is far behind the chemical modification. Nevertheless, these conservative approaches are very time- and cost-consuming procedures with high failure rates.

Nanoformulation is currently one of the most progressive fields of the pharmaceutical industry to increase solubility, bioavailability as well as reduce food and side effects of such active ingredients.

Nanoformulation is the reduction of particles size down to below 200 nm. The reduction of particle size leads to significantly increased dissolution rate of the active ingredients, which in turn can lead to increases in bioavailability.

There are two main approaches to making nanoparticles: “top-down” and “bottom-up” technologies. The conventional top-down approach basically relies on mechanical attrition to render large crystalline particles into nanoparticles. The bottom-up approach relies on controlled precipitation. The process involves dissolving the drugs in a solvent and precipitation in a controlled manner to nanoparticles through addition of an antisolvent.

Technologies relying on milling (top-down) or high-pressure homogenization (mixture of uncontrolled-bottom-up and top-down) are cost and time consuming methods. Both processes require high energy. This means that a large number of active compounds cannot be nanoformulated with these approaches due to heat induced active form conversion. For example, salt or active compounds with low melting point cannot be milled or high-pressure homogenized. The scale-up (industrial applicability) of the high energy processes are difficult and limited in many cases. These technologies target only late stage formulation or reformulation of poorly soluble active compounds to improve their efficiency.

Ezetimibe composition and the process for its preparation are described for example in WO/2005/009955, WO/2006/137080, WO/2009/074286, WO/2008/074723, WO/2005/062897 and WO/2009/150038 patent applications.

Nanoparticle compositions are described for example, in WO 2008074723, US 20080085315 and US 20060160785.

The nanoparticles of active pharmaceutical ingredients can be made using, for example, milling, homogenization, precipitation techniques, or supercritical fluid techniques, as is known in the art. Methods of making nanoparticulate compositions are also described in U.S. Pat. No. 5,718,388, U.S. Pat. No. 5,862,999, U.S. Pat. No. 5,665,331, U.S. Pat. No. 5,543,133, U.S. Pat. No. 5,534,270 and US 20070275075.

### B. Background Regarding Ezetimibe

Ezetimibe is in a class of lipid-lowering compounds that selectively inhibits the intestinal absorption of cholesterol and related phytosterols. The chemical name of Ezetimibe is 1-(4-fluorophenyl)-3(R)-[3-(4-fluorophenyl)-3(S)-hydroxypropyl]-4(S)-(4-hydroxyphenyl)-2-azetidinone. The empirical formula is C₂₄H₂₁F₂NO₃. Its molecular weight is 409.4 and its structural formula is:

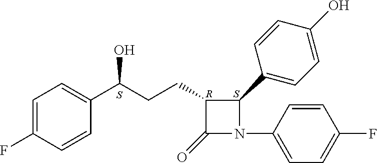

Ezetimibe is a white, crystalline powder that is freely to very soluble in ethanol, methanol, and acetone and practically insoluble in water. Ezetimibe has a melting point of about 163° C. and is stable at ambient temperature. ZETIA is available as a tablet for oral administration containing 10 mg of Ezetimibe and the following inactive ingredients: croscarmellose sodium NF, lactose monohydrate NF, magnesium stearate NF, microcrystalline cellulose NF, povidone USP, and sodium lauryl sulphate NF.

Ezetimibe is available as 10 mg tablets in most markets. A combination preparation Ezetimibe/simvastatin, which combines Ezetimibe with a statin, is also available.

### Pharmacokinetics

### Absorption

After oral administration, Ezetimibe is absorbed and extensively conjugated to a pharmacologically active phenolic glucuronide (Ezetimibe-glucuronide). After a single 10 mg dose of ZETIA to fasted adults, mean Ezetimibe peak plasma concentrations (Cₘₐₓ) of 3.4 to 5.5 ng/mL were attained within 4 to 12 hours (tₘₐₓ). Ezetimibe-glucuronide mean Cₘₐₓ values of 45 to 71 ng/mL were achieved between 1 and 2 hours (tₘₐₓ). There was no substantial deviation from dose proportionality between 5 and 20 mg. The absolute bioavailability of Ezetimibe cannot be determined, as the compound is virtually insoluble in aqueous media suitable for injection. The bioavailability is estimated to be between 35-65%.

### Effect of Food on Oral Absorption

Concomitant food administration (high-fat or non-fat meals) had no effect on the extent of absorption of Ezetimibe when administered as ZETIA 10-mg tablets. The Cₘₐₓ value of

Ezetimibe was increased by 38% with consumption of high-fat meals. ZETIA can be administered with or without food.

### Distribution

Ezetimibe and Ezetimibe-glucuronide are highly bound (>90%) to human plasma proteins.

### Side Effects

Side-effects include gastro-intestinal disturbances; headache, fatigue; myalgia; rarely arthralgia, hypersensitivity reactions (including rash, angioedema, and anaphylaxis), hepatitis; very rarely pancreatitis, cholelithiasis, cholecystitis, thrombocytopenia, raised creatine kinase, myopathy, and rhabdomyolysis.

All patients starting therapy with Ezetimibe should be advised of the risk of myopathy and told to report promptly any unexplained muscle pain, tenderness or weakness. The risk of this occurring is increased when taking certain types of medication. Patients should discuss all medication, both prescription and over-the-counter, with their physician.

Because of the insolubility of Ezetimibe in water and in biological relevant media, there is a need in the art to increase its solubility/dissolution rate/enhance bioavailability/accelerate the onset of action and reduce the dosage in order to overcome the problems associated with prior conventional Ezetimibe formulations. These problems can be solved by novel nanostructured particle formation of Ezetimibe characterized by increased solubility/dissolution rate, higher permeability, bioequivalence or higher Cₘₐₓ and longer duration of action compared to reference active compound and/or to the marketed drug described in the present invention. The present invention satisfies this need.

### DESCRIPTION OF THE INVENTION

The present invention describes the nanoparticulate Ezetimibe composition with enhanced solubility/dissolution rate/permeability/bioequivalent or increased bioavailability and absorption and longer duration of action.

The invention comprises a stable nanostructured Ezetimibe composition comprising:
(a) nanostructured Ezetimibe having an average particle size of less than about 400 nm; and
(b) at least one stabilizer and
(c) optionally any additional stabilizer for steric and electrostatic stabilization
   wherein the composition of the invention is prepared preferably in a continuous flow reactor, more preferable in microfluidic based continuous flow reactor.

In the composition of the invention can be used in a phase selected from a crystalline phase, an amorphous phase, a semi-crystalline phase, a semi-amorphous phase, a co-crystal and mixtures thereof.

As exemplified in the examples below, not every combination of stabilizers will result in a stable nanostructured particle formation. It was discovered, that stable nanostructured particles of Ezetimibe and its pharmaceutically acceptable salts can be made by continuous flow precipitation method using selected stabilizers.

The expression Ezetimibe is generally used for Ezetimibe and its pharmaceutically acceptable salts.

For the preparation of the composition of the invention stabilizers include nonionic, anionic, cationic, ionic polymers/surfactants and zwitterionic surfactants can be used. Combinations of more than one stabilizer can also be used in the invention. Useful stabilizers which can be employed in the invention include, but are not limited to known organic and inorganic pharmaceutical excipients. Such excipients include various polymers, low molecular weight oligomers, natural products, and surfactants.

Representative examples of stabilizers include hydroxypropyl methylcellulose, hydroxypropylcellulose, polysaccharide such as mannitol, sorbitol, polyvinylpyrrolidone (like Luviskol®), graft copolymer comprised of polyethylene, glycol, polyvinylcaprolactam and polyvinylacetate (Soluplus®); sodium lauryl sulfate, gelatin, cetostearyl alcohol, polyethylene glycols, acetic acid, ethenyl ester polymer with 1-ethenyl-2-pyrrolidinone (PVP/VA copolymers), sodium dodecyl benzene sulfonate, tocopheryl polyethylene glycol succinates, urea, citric acid, sodium-acetate, polyethoxylated castor oils and its derivateives, polyoxyethylene stearates, methylcellulose, hydroxyethylcellulose, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics, which are block copolymers of ethylene oxide and propylene oxide, Lutrol®); poloxamines (e.g., Tetronic, also known as Poloxamine, which is a tetrafunctional block copolymer derived from sequential addition of propylene oxide and ethylene oxide to ethylenediamine, D-alfa-Tocopherol polyethylene glycol 1000 succinate, poly(2-ethyl-2-oxazoline), poly (methyl vinyl ether), random copolymers of vinyl pyrrolidone and vinyl acetate, such as Plasdone S630 and the like.

Examples of useful ionic stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), benzalkonium chloride, hexadecyltrimethylammonium bromide, hexyldesyltrimethylammonium bromide (HDMAB), and polyvinylpyrrolidone-2-dimethylaminoethyl methacrylate dimethyl sulfate.

Advantages of the composition of the invention include, but are not limited to: (1) it has bioequivalent pharmacokinetic profile or higher Cₘₐₓ, higher AUC (bioavailability) and longer duration of action compared to the reference and/or marketed drugs; (2) it has increased solubility of Ezetimibe and (3) increased rate of dissolution for Ezetimibe nanostructured particles as compared to conventional forms of the same active compound; (4) it has significantly increased permeability.

Another aspect of the invention is a process for the preparation of nanostructured Ezetimibe comprising mixing an appropriate solvent of Ezetimibe with a solution of one or more stabilizers in a continuous flow reactor, preferable in a microfluidic continuous flow reactor.

Preferably the process for the preparation of the composition of the invention is carried out by (1) dissolving Ezetimibe and optionally one or more stabilizer in a suitable solvent; (2) adding the formulation from step (1) to a solution comprising at least one stabilizer; and (3) precipitating the formulation from step (2).

Preferably the process for the preparation of the composition of the invention is carried out by (1) dissolving Ezetimibe and one or more stabilizer(s) in a suitable solvent; (2) adding the formulation from step (1) to a solution from step (1) to a solvent comprising optionally one or more stabilizer(s); and (3) precipitating the formulation from step (2).

The process is carried out by (a) using two different solvents miscible with each other, where Ezetimibe is soluble only in one of them with the restriction that the applied stabilizer(s) is soluble in the solvents used. Such solvents may be dimethyl-sulfoxyde, methanol, ethanol, isopropanol, acetonitrile, tetrahydrofuran, acetone and pyridine preferably.

As a continuous flow reactor preferable a microfluidics based continuous flow reactor, described in the publication Microfluid Nanofluid DOI 10.1007/s10404-008-0257-9 by I. Hornyak, B. Borcsek and F. Darvas, is used.

The particle size of the nanostructured Ezetimibe may be influenced by the solvents used, the flow rate and the Ezetimibe—stabilizer ratio.

Another aspect of the invention is directed to the good/instantaneous redispersibility of solid nanostructured form of Ezetimibe in water and in biologically relevant mediums, e.g.; physiological saline solution, pH=2.5 HCl solution, FessiF and FassiF meadia.

Another aspect of the invention is a pharmaceutical composition comprising a stable nanostructured Ezetimibe or composition of them according to the invention and optionally pharmaceutically acceptable auxiliary materials.

The pharmaceutical composition of the invention can be formulated: (a) for administration selected from the group consisting of oral, pulmonary, rectal, colonic, parenteral, intracisternal, intravaginal, intraperitoneal, ocular, otic, local, buccal, nasal, and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

The compositions can be formulated by adding different types of excipients for oral administration in solid, liquid, local (powders, ointments or drops), or topical administration, and the like.

A preferred dosage form of the invention is a solid dosage form, although any pharmaceutically acceptable dosage form can be utilized.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alginates, gelatin, 35 polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Besides such inert diluents, the composition can also include adjuvants, such as wetting agents, emulsifying and suspending agents, sweetening, flavoring, and perfuming agents.

The pharmaceutical compositions of the invention show increased solubility/dissolution rate, higher permeability, bioequivalence or higher Cₘₐₓ and faster onset of action compared to reference active compound and/or to the marketed drug described in the present invention.

The present invention is also directed to methods of lowering cholesterol level using the novel Ezetimibe nanoparticles disclosed herein.

### A. Preferred Characteristics of Ezetimibe Nanoparticles of the Invention

### 1. Increased Solubility and Dissolution Rate of Nanostructured Ezetimibe

Nanostructured Ezetimibe compositions of the invention have increased solubility and dissolution profile due to the decreased particles size and nanostructured particle formation.

### Example 1

### Determination of Cₘₐₓ

The solubility of nanostructured Ezetimibe compared to the reference form of the active compound was determined in distilled water and 0.05% SDS solution by UV-VIS measurements (Thermo Genesys 10S spectrophotometer) at 248 nm wavelength and room temperature. The redispersed sample was filtered by 100 nm disposable syringe filter.

The solubility of reference Ezetimibe was under the detection limit in both applied media. The solubility of the nanostructured Ezetimibe was 0.06 mg/mL in distilled water and 0.6 mg/mL in 0.05% SDS solution, which indicates the increased solubility of nanostructured Ezetimibe.

### 2. Instantaneous Wettability and Dissolution of Nanostructured Ezetimibe

For the Ezetimibe to dissolve, its surface has first to be wetted by the surrounding fluid. The nanostructural forms possess a chemically randomized surface which expresses hydrophobic and hydrophilic interactions due to the nature of the stabilizer/(s) and active pharmaceutical ingredient, which can lead to improved wettability. If the surface of the Ezetimibe nanoparticles of the invention is functionalized by hydrophilic groups/stabilizer(s), a higher degree of hydrophility causes faster surface wetting and faster dissolution compared to the original crystalline form. This advanced property of the Ezetimibe nanoparticles of the present invention is supported by the results of the redispersibility test also. Due to the bigger surface area of the nanostructured Ezetimibe and the hydrophilic groups of the stabilizer(s) the surface wetting is instantaneous compared to the crystalline forms.

### Example 2

### Visual Observation of the Wettability and Dissolution of Nanostructured Ezetimibe

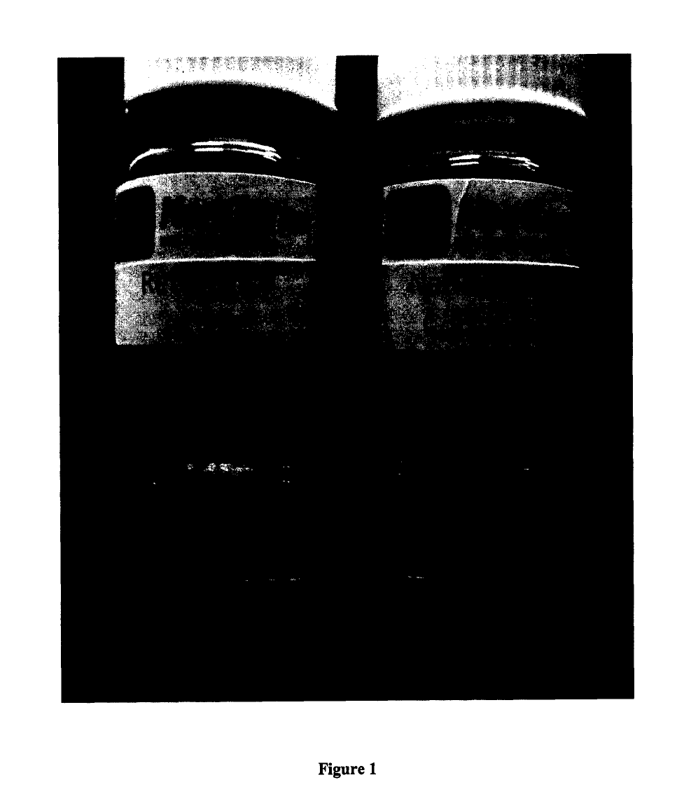

The wettability and dissolution of nanostructured Ezetimibe powder was performed by dispersing 5 mg nanosized Ezetimibe powder in 5 mL distillate water. After adding distilled water addition the vial was gently shaken by hand resulting colloid dispersion of nanostructured Ezetimibe particles as it is demonstrated in FIG. 1.

FIG. 1.: Instantaneous wettability and dissolution of nanostructured Ezetimibe in distillate water

An additional feature of the nanostructured Ezetimibe compositions of the present invention is that the dried nanoparticles stabilized by stabilizer(s) can be redispersed instantaneously or by the addition of traditional redispersants such as mannitol, sucrose.

### Example 3

### Redispersibility Test of Nanostructured Ezetimibe

Redispersibility test was performed by redispersing nanostructured Ezetimibe powder in distillate water. 5 mg freeze dried nanostructured Ezetimibe was redispersed in 5 mL distillate water under gentle stirring. The particles size of the redispersed sample was measured by DLS method (Nanotrac instrument, Mictrotrac Co., USA).

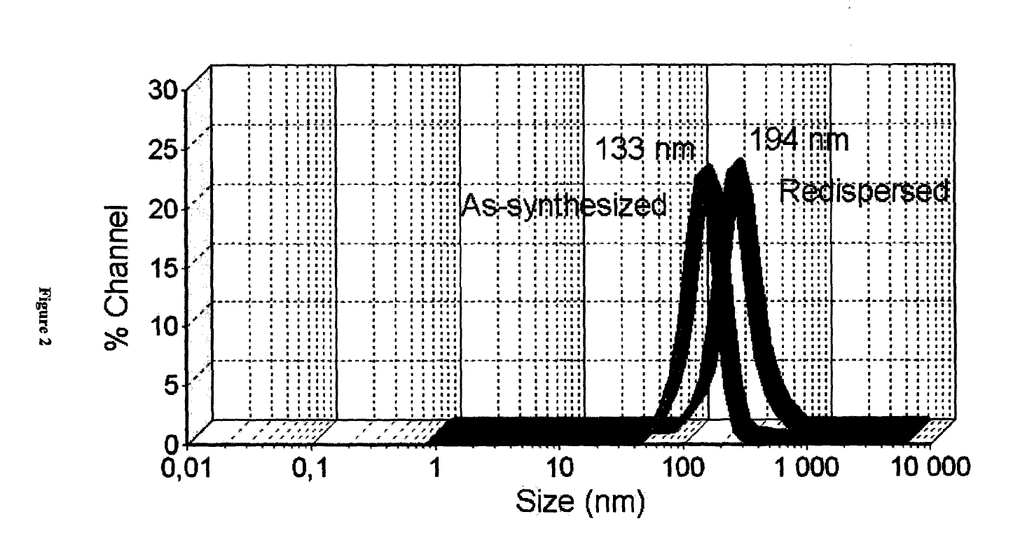

The mean particle size of redispersed nanostructure Ezetimibe (intensity-based average) was d=194 nm, while d(90) value was 303 nm as demonstrated in FIG. 2.

The significant benefit which can be obtained by nanoformulation is that the Ezetimibe nanoparticles of the present invention can be redispersed after the drying/solid formulation procedure having similar average particle size. Having the similar average particles size after the redispersion, the dosage form cannot lose the benefits afforded by the nanoparticle formation. A nanosize suitable for the present invention is an average particle size of less than about 400 nm.

FIG. 2: Size and size distribution of the Ezetimibe the nanoparticles before (as-synthesized) and after its redispersion.

### 3. Crystallographic Structure of Nanostructured Ezetimibe Composition of the Invention

The chemical stability of solid drugs is affected by the crystalline state of the drug. Many drug substances exhibit polymorphism. Each crystalline state has different chemical reactivity. The stability of drugs in their amorphous form is generally lower than that of drugs in their crystalline form, because of the higher free-energy level of the amorphous state. Decreased chemical stability of solid drugs brought about by mechanical stresses such as grinding is to a change in crystalline state. The chemical stability of solid drugs is also affected by the crystalline state of the drug through differences in surface area. For reaction that proceeds on the solid surface of the drug, an increase in the surface area can increase the amount of drug participating in the reaction.

### Example 4

### Crystallographic Structure Determination by Powder X-Ray Diffraction Analysis

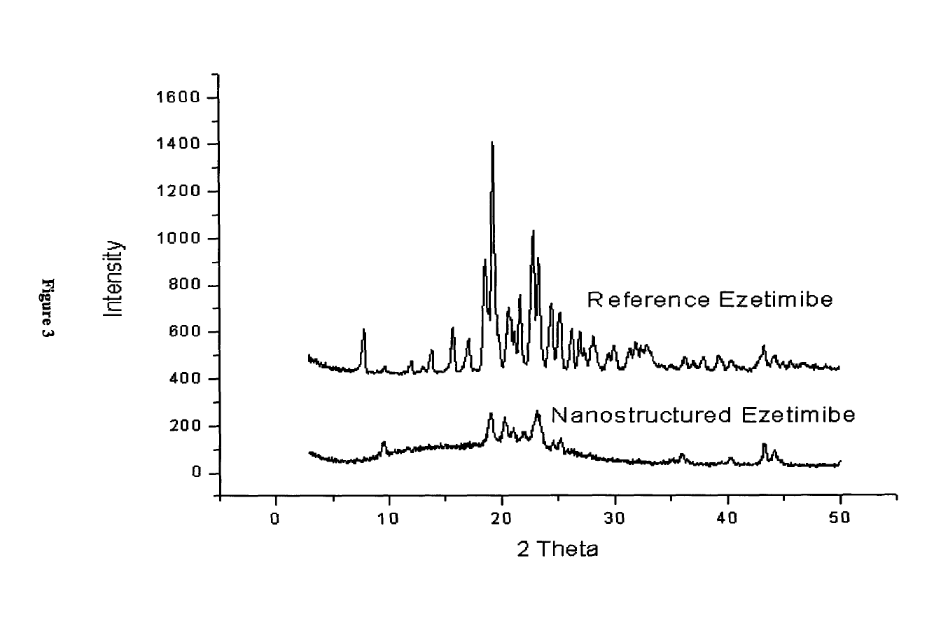

The structure of the Ezetimibe nanoparticles was investigated by X-ray diffraction analysis (Philips PW1050/1870 RTG powder-diffractometer). The nanostructured Ezetimibe composition showed crystalline character, however the characteristic diffractions of the reference crystalline Ezetimibe could not be found. The X-ray diffractograms are demonstrated in FIG. 3.

FIG. 3: X-ray diffractograms of reference Ezetimibe and nanostructured Ezetimibe of the invention

### 4. Enhanced In Vitro Biological Performance of Nanostructured Ezetimibe

In order to demonstrate the improved pharmacokinetic properties of the novel nanostructured Ezetimibe PAMPA permeability measurements were performed.

### Example 5

### PAMPA Permeability Measurements

Reference Ezetimibe and solid nanostructured Ezetimibe was redispersed in distilled water and permeability was measured across and artificial membrane composed of dodecane with 20% soy lecithin. The sample containing the reference compound was a suspension of crystals visible by the naked eye, while the nanostructured sample was an opalescent colloid solution. The receiver compartment was phosphate buffered saline with 1% sodium lauryl sulphate.

The PAMPA permeability of the reference compound could not be determined because the concentration of Ezetimibe in the receiver compartment was under the detection limit of the method used (UV spectroscopy at 248 nm). The PAMPA permeability of the novel nanostructured formula was 1.417*10⁻⁶+/−0.052*10⁻⁶ cm/s.

Based on the improved PAMPA permeability of nanostructured Ezetimibe when compared to the unformulated compound we concluded that nanostructured Ezetimibe could have superior pharmacokinetic properties (higher bioavailability, higher cₘₐₓ) when compared to the unformulated form. Although the target of the compound is in the small intestine Ezetimibe and its glucuronide metabolite is involved in extensive enterohepatic circulation. Increasing the absorption and bioavailability of Ezetimibe should result in higher concentrations of the parent compound and its metabolite in the enterohepatic circulation resulting in better pharmacological response and longer duration of action.

### B. Compositions

The nanoparticles of Ezetimibe and its compositions according to the invention have an average particle size of less than about 400 nm. The stable nanostructured particles of the present invention are characterized by increased solubility, dissolution rate/increased permeability and bioequivalent or superior biological performance compared to the reference and marketed forms.

The stabilizers preferably are associated or interacted with the Ezetimibe and its pharmaceutically acceptable salts, but do not chemically react with the Ezetimibe or themselves.

The nanoparticles of Ezetimibe of the invention can be prepared by solvent-antisolvent nano-precipitation methods using stabilizer(s).

### Particle Size of the Nanostructured Ezetimibe Particles

The invention contains Ezetimibe nanoparticles, which have an average particle size of less than about 400 nm as measured by dynamic light scattering method.

By “an average particle size of less than about 400 nm” it is meant that at least 50% of the Ezetimibe and its pharmaceutically acceptable salts have a particle size of less than the average, by number/intensity, i.e., less than about 400 nm, etc., when measured by the above-noted technique.

### Example 6

### Process for Producing Nanostructured Ezetimibe Stable DMSO/Water Based Colloid Solution

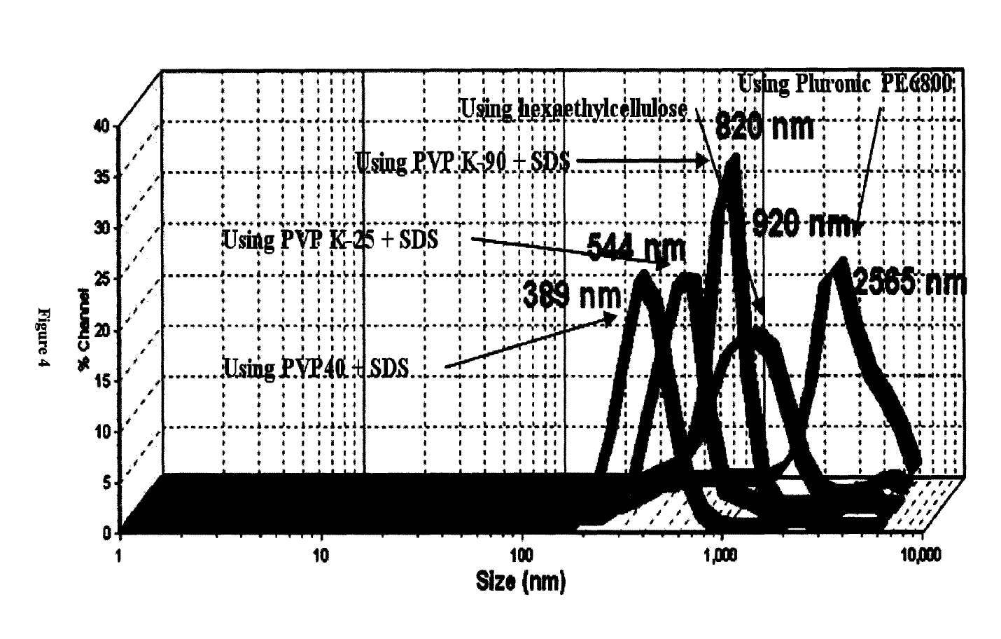

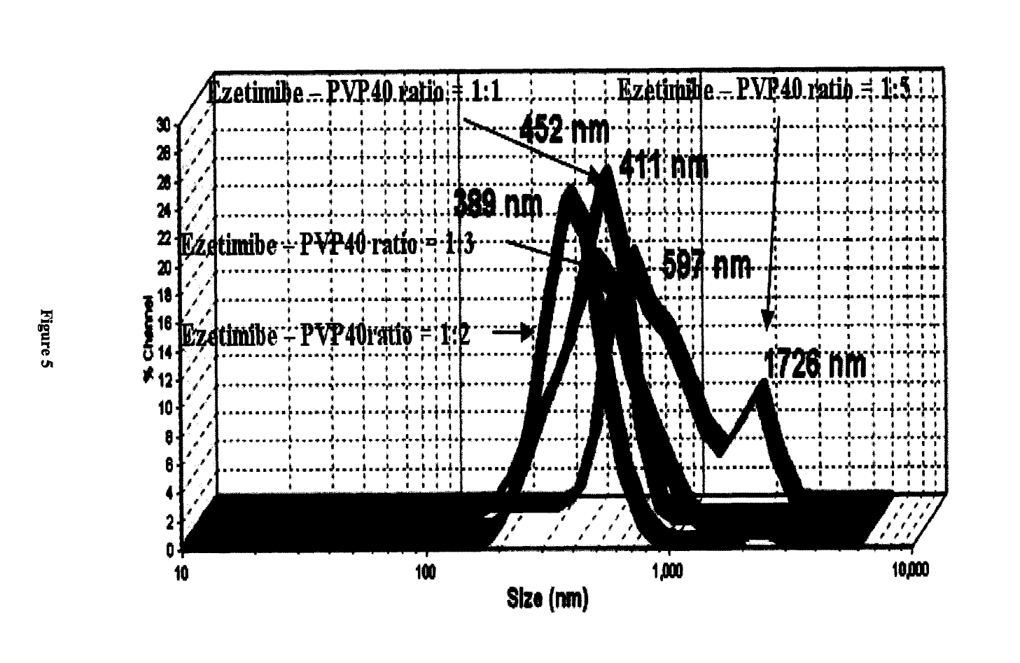

During the experiments Ezetimibe nanoparticles were prepared in a microfluidic based continuous flow reactor. As a starting solution, 800 mg Ezetimibe, 320 mg sodium dodecyl sulphate and 1600 mg polyvinylpyrrolidone, PVP40 dissolved in 100 mL DMSO was used. The prepared solution was passed into the reactor unit with 1 mL/min flow rate using a feeding unit. Meanwhile, using a second feeding unit, distilled water was passed into a mixing unit with 1.2 mL/min flow rate, where it was mixed with the solution containing Ezetimibe coming from the first reactor unit. The nanoparticles are continuously produced at atmospheric pressure due to the chemical precipitation by water passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates; pressure and the types of the stabilizers (see FIG. 4.). The particles size and size distribution of the Ezetimibe particles can be controlled by the amount the stabilizer(s) (PVP40) as it is show in FIG. 5. The particles size of the Ezetimibe particle was 389 nm in the best case.

FIG. 4.: Particle size and size distribution of Ezetimibe nanoparticles using different stabilizers

FIG. 5.: Effect of the stabilizer concentration on the particle size and size distribution of Ezetimibe nanoparticles

### Example 7

### Process for Producing Nanostructured Ezetimibe Stable Tetrahydrofuran/Water Based Colloid Solution

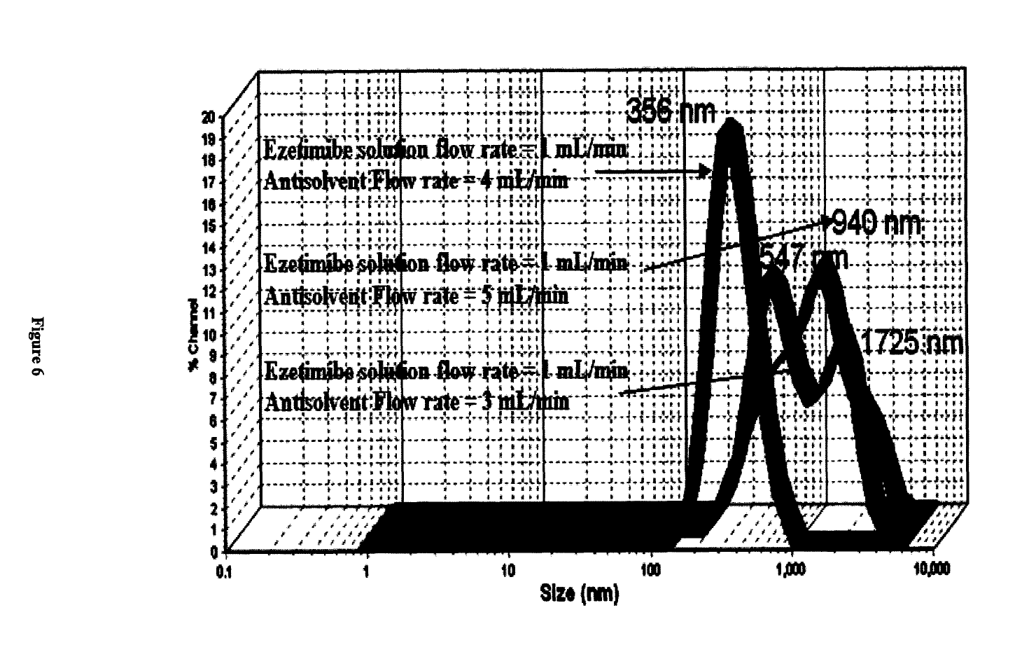

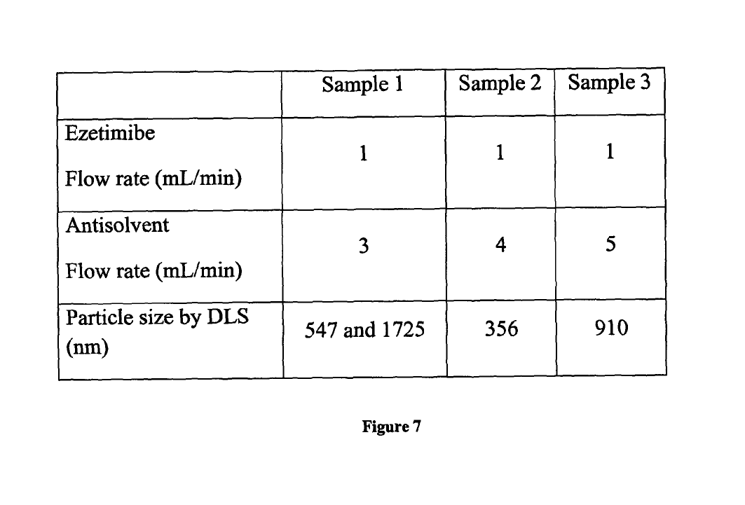

During the experiments Ezetimibe nanoparticles were prepared in a microfluidic based continuous flow reactor. As a starting solution, 200 mg Ezetimibe and 800 mg Pluronic PE6800 dissolved in 100 mL Tetrahydrofuran was used. The prepared solution was passed into the reactor unit with 1 mL/min flow rate using a feeding unit. Meanwhile, using a second feeding unit, distilled water was passed into a mixing unit with 4 mL/min flow rate, where it was mixed with the solution containing Ezetimibe coming from the first reactor unit. The nanoparticles are continuously produced at atmospheric pressure due to the chemical precipitation by water passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates (see FIG. 6.). The particles size and size distribution of the Ezetimibe particle was 356 nm in the best case (see FIG. 7.).

FIG. 6.: Particle size and size distribution of Ezetimibe nanoparticles using different flow rates.

FIG. 7.: Effect of the flow rates on the particle size of Ezetimibe

### Example 8

### Process for Producing Solid Nanostructured Ezetimibe Composition

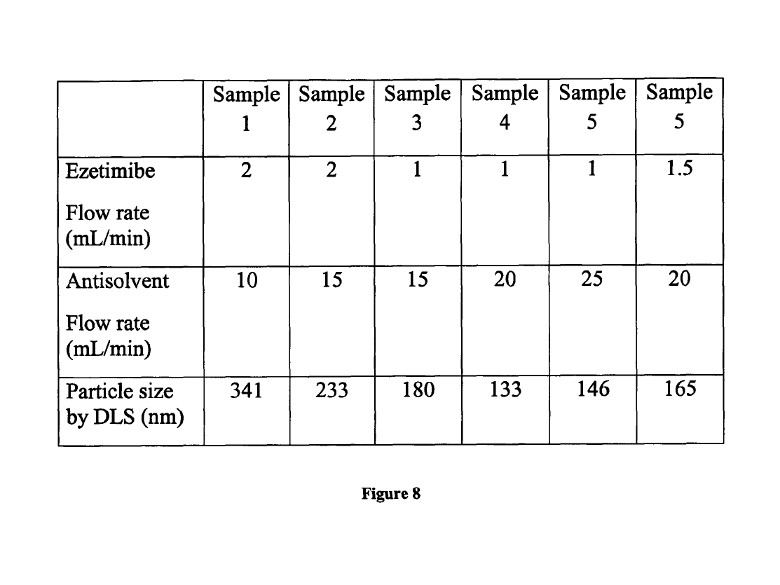

During the experiments Ezetimibe nanoparticles were prepared in a microfluidic based continuous flow reactor. As a starting solution, 1000 mg Ezetimibe, 1000 mg Pluronic PE6800 and 4000 mg Luviscol VA64 were dissolved in 100 mL MeOH was used. The prepared solution was passed into the reactor unit with 1 mL/min flow rate using a feeding unit. Meanwhile, using a second feeding unit, dissolved in distilled water was passed into a mixing unit with 20 mL/min flow rate, where it was mixed with the solution containing Ezetimibe coming from the first reactor unit. The nanoparticles are continuously produced at atmospheric pressure at 15° C. due to the chemical precipitation by 0.5 mg/mL Lutrol F127 solution passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates. The particles size and size distribution of the Ezetimibe particle was 133 nm in the best case (see FIG. 8.). The resulted colloid was then freeze dried in order to obtain the nanostructured Ezetimibe in solid form.

FIG. 8: Particle size and size distribution of Ezetimibe nanoparticles using different flow rates

1-10. (canceled)

11. A stable nanostructured Ezetimibe composition comprising:
(a) nanostructured Ezetimibe or its pharmaceutically acceptable salt having an average particle size of less than about 400 nm;
(b) at least one stabilizer;
wherein the composition is prepared in a continuous flow reactor.

12. The stable nanostructured Ezetimibe composition according to claim 11 wherein the composition is prepared in a microfluidic based continuous flow reactor.

13. A stable nanostructured Ezetimibe composition according to claim 11 further comprising an additional stabilizer for steric and electrostatical stabilization.

14. The composition according to claim 11, wherein the stabilizer is selected from the group of non-ionic, anionic, cationic polymers/surfactants, and zwitterionic surfactants, or combinations thereof.

15. The composition according to claim 11 wherein the stabilizer is selected from the group of cellulose and its derivatives, polysaccharides such as mannitol, sorbitol, polyvinylpyrrolidone such as Luviskol®, graft copolymer comprised of polyethylene, glycol, polyvinylcaprolactam and polyvinylacetate (Soluplus®); sodium lauryl sulfate, gelatin, cetostearyl alcohol, polyethylene glycols, acetic acid, ethenyl ester polymer with 1-ethenyl-2-pyrrolidinone (PVP/VA copolymers), sodium dodecyl benzene sulfonate, tocopheryl polyethylene glycol succinates, urea, citric acid, sodium-acetate, polyethoxylated castor oils and its derivateives, polyoxyethylene stearates, methylcellulose, hydroxyethylcellulose, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics, which are block copolymers of ethylene oxide and propylene oxide, Lutrol®); poloxamines (e.g., Tetronic, also known as Poloxamine, which is a tetrafunctional block copolymer derived from sequential addition of propylene oxide and ethylene oxide to ethylenediamine, D-alfa-Tocopherol polyethylene glycol 1000 succinate, poly(2-ethyl-2-oxazoline), poly (methyl vinyl ether), random copolymers of vinyl pyrrolidone and vinyl acetate, such as Plasdone S630.

16. The composition according to claim 11 wherein the stabilizer is selected from the group of a polyvinyl alcohol, block copolymers of ethylene oxide and propylene oxide, polyvinylpyrrolidone, sodium acetate, polysaccharides, graft copolymer comprised of polyethylene, glycol, polyvinylcaprolactam and polyvinylacetate.

17. A process for the preparation of nanostructured Ezetimibe composition according to claim 11, comprising mixing the solution of Ezetimibe or its pharmaceutically acceptable salt and at least one stabilizer with an other solvent preferably water containing optionally at least one stabilizer in a continuous flow reactor, preferably in a microfluidic based continuous flow reactor.

18. The process according to 17, comprising (1) dissolving Ezetimibe or its pharmaceutically acceptable salt and at least one stabilizer in a suitable solvent; (2) adding the formulation from step (1) to a solvent optionally comprising one or more stabilizers; and (3) precipitating the formulation from step (2).

19. A pharmaceutical composition comprising a nanostructured composition according to claim 11 together with pharmaceutically acceptable auxiliary materials, preferably in the form of oral, pulmonary, rectal, parenteral, intracisternal, intravaginal, intraperitoneal, ocular, otic, local, buccal, nasal or topical administration.

20. Use of nanostructured composition according to claim 11 or a pharmaceutical composition comprising a nanostructured composition according to claim 11 for preparation of a medicament.

21. Use of nanostructured composition according to claim 11 or a pharmaceutical composition comprising a nanostructured composition according to claim 11 for the treatment to lower cholesterol levels.

22. The use according to claim 20, wherein the composition has
a solubility at least about 0.06 mg/ml in water and 0.6 mg/mL in SDS solution,
in vitro permeability through artificial membrane at least 1.4*10⁻⁶ cm/s,
instantaneous redispersibility in a physiological medium,
reduced or eliminated fed/fasted effect,
at least bioequivalent absorption in human gastrointestinal tract compared to the reference or marketed compound,
faster onset of action,
for decreasing the dosage used.

23. A method for the treatment of a subject in need thereof by administering to the subject an effective amount of nanostructured Ezetimibe composition according to claim 11 or the pharmaceutical composition comprising a nanostructured composition according to claim 11.

24. The method according to claim 23 for the treatment to lower cholesterol levels.

25. The method according to claim 23, wherein the composition has
a solubility at least about 0.06 mg/ml in water and 0.6 mg/mL in SDS solution,
in vitro permeability through artificial membrane at least 1.4*10⁻⁶ cm/s,
instantaneous redispersibility in a physiological medium,
reduced or eliminated fed/fasted effect,
at least bioequivalent absorption in human gastrointestinal tract compared to the reference or marketed compound,
faster onset of action,
for decreasing the dosage used.
